(19) United States
(12) Patent Application Publication
(10) Pub. No.: US 2017/0352910 A1
(43) Pub. Date: Dec. 7, 2017

(54) ELECTROLYTE FOR LITHIUM SECONDARY BATTERY AND LITHIUM SECONDARY BATTERY COMPRISING SAME

(71) Applicant: Samsung SDI Co., Ltd., Yongin-si, Gyeonggi-do (KR)

(72) Inventors: Joo Hwan KOH, Seoul (KR); Jong Ho JEON, Daejeon (KR); Jin Hee KIM, Suwon-si (KR); Sung Nim JO, Seoul (KR); Tae Hwan YU, Seoul (KR); Jung Joo CHO, Hwaseong-si (KR)

(21) Appl. No.: 15/538,955

(22) PCT Filed: Jul. 27, 2015
(86) PCT No.: PCT/KR2015/007786

§ 371 Date: Jun. 22, 2017

(30) Foreign Application Priority Data
Dec. 22, 2014 (KR) 10-2014-0185908

(51) Int. Cl. H01M 10/052; H01M 10/0567; H01M 10/0568; C07C 211/62; C07C 301/00; C01D 15/00; C01B 25/455; C07C 305/00; C07C 69/01

(52) U.S. Cl. CPC H01M 10/052; H01M 10/0568; H01M 10/0567; C07C 211/62; C07C 69/01; C01D 15/005; C01B 25/455; C07C 305/00; C07C 301/00

(57) ABSTRACT

Provided are an electrolyte solution for a lithium secondary battery, and a lithium secondary battery including the electrolyte solution. The electrolyte solution for a lithium secondary battery includes a lithium salt, an organic solvent, and further quaternary ammonium hexafluorophosphate as a solid salt.

### TECHNICAL FIELD

The present disclosure relates to an electrolyte solution for a lithium secondary battery, and a lithium secondary battery including the electrolyte solution, and more particularly, to an electrolyte solution that may include a solid salt with an ammonium cation and a hexafluorophosphate anion as an additive to improve high-temperature charge and discharge efficiency and lifetime characteristics of a lithium secondary battery, and a lithium secondary battery including the electrolyte solution.

### BACKGROUND ART

Along with technical development and increasing demand for mobile devices, demand for secondary batteries as an energy source is rapidly increasing. In particular, currently lithium secondary batteries having high energy density, high working voltage, long cycle lifetime, and low self-discharge rate are widely commercially available.

Such a lithium secondary battery has a structure with an electrolyte assembly impregnated with an electrolyte solution containing a lithium salt, the electrolyte assembly including a positive electrode and a negative electrode that are obtained by coating electrode current collectors with respective positive and negative active materials and are separated from one another by a porous separator. During charging, lithium ions released from the positive active material are intercalated into a negative active material layer. During discharging, the lithium ions released from the negative active material layer are intercalated into the positive active material. The electrolyte solution serves as a migration medium of lithium ions between the negative electrode and the positive electrode.

In general, an electrolyte solution may include an organic solvent and an electrolyte salt. For example, a widely used electrolyte solution may consist of a mixed solvent of a high-dielectric cyclic carbonate such as propylene carbonate, or ethylene carbonate, and a low-viscosity chain carbonate such as diethylcarbonate, ethylmethylcarbonate, or dimethylcarbonate, and a lithium salt such as LiPF₆, LiBF₄, LiClO₄ added to the mixed solution.

Lithium-containing halide salts such as a lithium-containing fluoride salt or a lithium-containing chloride salt which may be used as the electrolyte salt are highly sensitive to moisture, generating a strong acid (HX, wherein X is F, Cl, Br, or I) by reaction with moisture during the manufacture of a battery or with moisture present in the battery. In particular, since LiPF₆ as a lithium salt is unstable at high temperature, its anions may be thermally decomposed, generating an acidic material such as hydrofluoric acid (HF). This acidic material may unavoidably accompany an undesirable side reaction within the battery.

For example, a solid electrolyte interphase (SEI) layer on a surface of the negative electrode may be vulnerable to damage due to strong reactivity of the hydrofluoric acid (HF), which may induce continuous regeneration of the SEI layer, and increase the thickness of the coated SEI layer of the negative electrode and an interfacial resistance of the negative electrode. As lithium fluoride (LiF) as a byproduct of the generation of hydrofluoric acid (HF) is adsorbed onto the surface of the positive electrode, an interfacial resistance of the positive electrode may also be increased. In addition, the strong acid (HX) may cause radical oxidation reaction within the battery, and consequently dissolution and degeneration of the electrode active materials. In particular, as transition metal cations included in a lithium metal oxide used as a positive active material are dissolved, the cations may be electrodeposited onto the negative electrode, forming an additional coating layer on the negative electrode, consequently further increasing the resistance of the negative electrode.

The SEI layer, which may be formed on the surface of the negative electrode by reaction of a polar non-aqueous carbonate solvent with lithium ions of the electrolyte solution during initial charging of a lithium secondary battery, may serve as a protective layer by inhibiting decomposition of the carbonate electrolyte solution to stabilize the battery. However, an SEI layer formed of only an organic solvent and a lithium salt may not be enough to consistently serve as a protective layer and thus may be gradually damaged during continuous charging and discharging of the battery or storage of the battery at high temperature in a fully charged state, due to increased electrochemical energy and heat energy. A side reaction of decomposing a surface of the negative active material exposed through the damaged SEI layer by reaction with the electrolyte solution solvent may continuously occur, leading to resistance increase of the negative electrode.

The electrode-electrolyte interfacial resistance may be increased by other various factors besides the above-described factors. Such an increased interfacial resistance may lead to deterioration in overall performance of the battery, including output characteristics. For example, an increased resistance of a battery may lead to change in average voltage of charging and discharging, i.e., an increase in average voltage of discharging and a reduction in average voltage of charging. Consequently, the charge and discharge efficiency, which is represented as a ratio of discharge capacity to charge capacity in charging and discharging with a constant current, may be reduced.

To address these drawbacks, patent document 1 (JP 1993-13088) discloses a method of improving resistance of a lithium secondary battery by adding vinylene carbonate (VC) to an electrolyte solution. However, this method may result in a coating layer having still high resistance, and is not satisfactory in terms of effect of resistance increase suppression.

Patent document 2 (KR 2012-0011209) discloses an electrolyte solution for a lithium secondary battery including an alkylene sulfate having a specific structure, an ammonium compound having a specific structure, and vinylene carbonate. According to this method, an SEI layer formed from the sulfate compound may have reduced resistance, thus improving low-temperature output characteristics of the battery, but without a marked improvement in charge and discharge efficiency or lifetime characteristics of the battery. Therefore, there still is a need for further improvement in this regard.

### PRIOR ART DOCUMENT

### Patent Document

(Patent document 1) JP 1993-13088
(Patent document 2) KR 2012-0011209 A

### DETAILED DESCRIPTION OF THE INVENTION

### Technical Problem

The present invention provides an electrolyte solution for a lithium secondary battery that includes a solid salt with an ammonium-based cation and a hexafluorophosphate anion as an additive, thereby improving high-temperature charge and discharge efficiency and lifetime characteristics.

The present invention provides a lithium secondary battery including the electrolyte solution.

### Technical Solution

According to an aspect of the present invention, an electrolyte solution for a lithium secondary battery includes a lithium salt and an organic solvent, wherein the electrolyte solution further includes a solid salt represented by Formula 1:

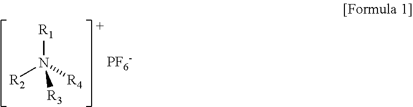

wherein, in Formula 1, R₁ to R₄ are each independently hydrogen, a halogen, or a C1 to C8 alkyl group.

In some embodiments, an amount of the solid salt may be in a range of 0.01 part to 5 parts by weight with respect to 100 parts by weight of a total weight of the lithium salt and the organic solvent.

In some embodiments, the solid salt represented by Formula 1 may be at least one selected from the group consisting of ammonium hexafluorophosphate, tetramethylammonium hexafluorophosphate, tetraethylammonium hexafluorophosphate, tetrapropylammonium hexafluorophosphate, tetrabutylammonium hexafluorophosphate, tetrahexylammonium hexafluorophosphate, tetraheptylammonium hexafluorophosphate, ethyltrimethylammonium hexafluorophosphate, triethylmethylammonium hexafluorophosphate, butyltrimethylammonium hexafluorophosphate, diethyldimethylammonium hexafluorophosphate, and dibutyldimethylammonium hexafluorophosphate.

According to another aspect of the present invention, a lithium secondary battery includes the electrolyte solution.

### Advantageous Effects of the Invention

As described above, according to the one or more embodiments, an electrolyte solution may further include a solid salt represented by Formula 1 with an ammonium-based cation and a hexafluorophosphate anion as an additive. A lithium secondary battery including the electrolyte solution may have improved high-temperature charge and discharge efficiency and lifetime characteristics.

### DESCRIPTION OF THE DRAWING

FIG. 1 is a comparative graph illustrating lifetime characteristics of lithium secondary batteries manufactured using electrolyte solutions of Example 1 and Comparative Example 1.

### MODE OF THE INVENTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

According to an aspect of the present disclosure, an electrolyte solution for a lithium secondary battery includes: a lithium salt and an organic solvent, wherein the electrolyte solution further includes a solid salt represented by Formula 1:

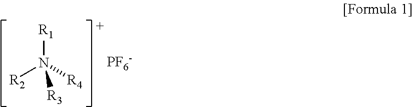

wherein, in Formula 1, R₁ to R₄ are each independently hydrogen, a halogen, or a C1 to C8 alkyl group.

A solid electrolyte interphase (SEI) layer refers to a film formed on an interface between a negative electrode and an electrolyte solution in a lithium secondary battery by reaction of the electrolyte solution with the negative electrode, which takes place as lithium ions deintercalated at the positive electrode migrate toward the negative electrode and are intercalated into the negative electrode during initial charging of the lithium secondary battery. The SEI layer may selectively pass only lithium ions, so that the intercalation of a large-molecular weight organic solvent of the electrolyte solution into the carbon negative electrode may be prevented, thus preventing a structural collapse of the negative electrode. The SEI layer may also inhibit side reaction between lithium ions and other materials during continuous charging and discharging processes. However, a SEI layer formed from a conventional carbonate-based organic solvent, a fluorine salt or other inorganic salts may have a weak, porous, and non-compact structure, failing to perform the above-described function.

In the electrolyte solution according to an embodiment, since the solid salt of Formula 1 used as an additive has a lower reduction potential than the organic solvent of the electrolyte solution, the solid salt may be reduced at the surface of the negative active material layer earlier than the organic solvent of the electrolyte solution during initial charging of a battery, forming a strong, dense SEI layer having good stability under high-temperature environments. Accordingly, the SEI layer formed from the solid salt may prevent a side reaction such as co-intercalation of the organic solvent of the electrolyte solution, for example, a carbonate organic solvent, into the negative active material layer or decomposition of the organic solvent on the surface of the negative electrode, thus improving charge and discharge efficiency and lifetime characteristics of the battery. Furthermore, since the solid salt may prevent decomposition and regeneration of the SEI layer, and thus suppress interfacial resistance increase of the negative electrode.

The amount of the solid salt may be from 0.01 part to 5.0 parts, and in some embodiments, 0.1 part to 3.0 parts by weight, with respect to 100 parts by weight of a total weight of the lithium salt and the organic solvent. When the amount of the solid salt is less than 0.01 part by weight, it may be difficult to form a SEI layer having good stability. On the other hand, when the amount of the solid salt exceeds 5.0 parts by weight, charge and discharge efficiency may be reduced.

In some embodiments, the solid salt of Formula 1 may be at least one selected from the group consisting of ammonium hexafluorophosphate, tetramethylammonium hexafluorophosphate, tetraethylammonium hexafluorophosphate, tetrapropylammonium hexafluorophosphate, tetrabutylammonium hexafluorophosphate, tetrahexylammonium hexafluorophosphate, tetraheptylammonium hexafluorophosphate, ethyltrimethylammonium hexafluorophosphate, triethylmethylammonium hexafluorophosphate, butyltrimethylammonium hexafluorophosphate, diethyldimethylammonium hexafluorophosphate, and dibutyldimethylammonium hexafluorophosphate. However, embodiments are not limited thereto.

In some embodiments, a concentration of the lithium salt in the electrolyte solution may be in a range of 0.6M to 2.0M, and in some embodiments, a range of 0.7M to 1.6M. When the concentration of the lithium salt is less than 0.6M, the electrolyte solution may have reduced conductivity and deteriorated performance. On the other hand, when the concentration of the lithium salt exceeds 2.0M, the electrolyte solution may have increased viscosity, consequently leading to reduced mobility of lithium ions. Any lithium salt commonly used in an electrolyte solution for a lithium secondary battery may be used. For example, anions of the lithium salt may be one selected from the group consisting of F⁻, Cl⁻, Br⁻, I⁻, NO₃⁻, N(CN)₂⁻, BF₄⁻, ClO₄⁻, PF₆⁻, (CF₃)₂PF₄⁻, (CF₃)₃PF₃⁻, (CF₃)₄PF₂⁻, (CF₃)₅PF⁻, (CF₃)₆P⁻, CF₃SO₃⁻, CF₃CF₂SO₃⁻, (CF₃SO₂)₂N⁻, (FSO₂)₂N⁻, CF₃CF₂(CF₃)₂CO⁻, (CF₃SO₂)₂CH⁻, (SF₅)₃C⁻, (CF₃SO₂)₃C⁻, CF₃(CF₂)₇SO₃⁻, CF₃CO₂⁻, CH₃CO₂⁻, SCN⁻, and (CF₃CF₂SO₂)₂N⁻.

The organic solvent in the electrolyte solution may be any organic solvent commonly used in an electrolyte solution for a lithium secondary battery. For example, the organic solvent may be an ether, an ester, an amide, a linear carbonate, or a cyclic carbonate, which may be used alone or a combination of at least two thereof.

Of these organic solvents, a cyclic carbonate, a linear carbonate, or a carbonate compound as a mixture of the forgoing two may be used. For example, the cyclic carbonate compound may be one selected from the group consisting of ethylene carbonate (EC), propylene carbonate (PC), 1,2-butylene carbonate, 2,3-butylene carbonate, 1,2-pentylene carbonate, 2,3-pentylene carbonate, vinylene carbonate, and a halide thereof, or a mixture of at least two thereof. For example, the linear carbonate compound may be one selected from the group consisting of dimethyl carbonate (DMC), diethyl carbonate (DEC), dipropyl carbonate, ethylmethylcarbonate (EMC), methylpropyl carbonate, and ethylpropyl carbonate, or a mixture of at least two thereof. However, embodiments are not limited thereto.

In particular, as cyclic carbonate organic solvents, ethylene carbonate and propylene carbonate which have high viscosity and high dielectric constant to dissociate a lithium salt in electrolyte may be used. For example, an electrolyte solution having a high electric conductivity prepared by mixing such a cyclic carbonate with a linear carbonate having low viscosity and low dielectric constant in an appropriate ratio may be used.

The ether as an organic solvent may be one selected from the group consisting of dimethyl ether, diethyl ether, dipropyl ether, methylethyl ether, methylpropyl ether, and ethylpropyl ether, or a mixture of at least two thereof. However, embodiments are not limited thereto.

The ester as an organic solvent may be one selected from the group consisting of methyl acetate, ethyl acetate, propyl acetate, methyl propionate, ethyl propionate, propyl propionate, γ-butyrolactone, γ-valerolactone, γ-caprolactone, σ-valerolactone, and ε-caprolactone, or a mixture of at least two thereof. However, embodiments are not limited thereto.

In some embodiments, the electrolyte solution for a lithium secondary battery may further include a conventionally known additive to form an SEI layer. For example, the additive to form an SEI layer may be vinylene carbonate, vinyl ethylene carbonate, fluoroethylene carbonate, a cyclic sulfite, a saturated sultone, a unsaturated sultone, or a noncyclic sulfone, which may be used alone or in a combination of at least two thereof. However, embodiments are not limited thereto.

The cyclic sulfite may be, for example, ethylene sulfite, methyl ethylene sulfite, ethyl ethylene sulfite, 4,5-dimethyl ethylene sulfite, 4,5-diethyl ethylene sulfite, propylene sulfite, 4,5-dimethyl propylene sulfite, 4,5-diethyl propylene sulfite, 4,6-dimethyl propylene sulfite, 4,6-diethyl propylene sulfite, or 1,3-butylene glycol sulfite. The saturated sultone may be, for example, 1,3-propane sultone, 1,4-butane sultone, or the like. The unsaturated sultone may be, for example, ethene sultone, 1,3-propene sultone, 1,4-butene sultone, or 1-methyl-1,3-propene sultone. The noncyclic sulfone may be, for example, divinylsulfone, dimethyl sulfone, diethyl sulfone, methylethyl sulfone, or methylvinyl sulfone.

The additive to form an SEI layer may be used in an appropriate amount, which may vary depending on a type of the additive. For example, the additive to form an SEI layer may be 0.01 part to 10 parts by weight with respect to 100 parts by weight of the electrolyte solution.

According to another aspect of the present disclosure, a lithium secondary battery includes an electrolyte solution according to any of the above-described embodiments.

The lithium secondary battery may be manufactured by injecting an electrolyte solution according to any of the above-described embodiments into an electrode assembly including a positive electrode, a negative electrode, and a separator between the positive electrode and the negative electrode.

The positive electrode and the negative electrode may each be manufactured by preparing a slurry by mixing positive or negative active material, a binder, and a conducting agent, coating a current collector such as aluminum foil with the slurry, and drying and pressing a resulting product.

The positive active material may be a lithium-containing transition metal oxide, for example, one selected from the group consisting of LiₓCoO₂ (wherein 0.5<x<1.3), LiₓNiO₂ (wherein 0.5<x<1.3), LiₓMnO₂ (wherein 0.5<x<1.3), LiₓMn₂O₄ (wherein 0.5<x<1.3), Liₓ(NiₐCo_{b}Mn_{c})O₂ (wherein 0.5<x<1.3, 0<a<1, 0<b<1, 0<c<1, and a+b+c=1), LiₓNi_{1-y}Co_{y}O₂ (wherein 0.5<x<1.3 and 0<y<1), LiₓCo_{1-y}Mn_{y}O₂ (wherein 0.5<x<1.3 and y≦1), LiₓNi_{1-y}Mn_{y}O₂ (wherein 0.5<x<1.3 and 0≦y<1), Liₓ(NiₐCo_{b}Mn_{c})O₄ (wherein 0.5<x<1.3, 0<a<2, 0<b<2, 0<c<2, and a+b+c=2), LiₓMn_{2-z}Ni_{z}PO₄ (wherein 0.5<x<1.3 and 0<z<2), LiₓMn_{2-z}Co_{z}O₄ (wherein 0.5<x<1.3 and 0<z<2), LiₓCoPO₄ (wherein 0.5<x<1.3) and LiₓFePO₄ (wherein 0.5<x<1.3), or a mixture of at least two thereof. The lithium-containing transition metal oxide may be coated with a metal such as aluminum (Al) or a metal oxide thereof. Also, a sulfide, a selenide, and a halide may be used in addition to these lithium-containing transition metal oxides.

The negative active material may be, for example, a carbonaceous material, a lithium metal, silicon, or tin from which lithium ions may generally intercalated and deintercalated. For example, the negative active material may be a metal oxide having a potential less than 2V with respect to lithium, such as TiO₂ or SnO₂. Examples of the carbonaceous material may include low-crystalline carbon and high-crystalline carbon. Examples of the low-crystalline carbon may include soft carbon and hard carbon. Examples of the high-crystalline carbon may include natural graphite, artificial graphite, Kishgraphite, pyrolytic carbon, mesophase pitch based carbon fiber, meso-carbon microbeads, mesophase pitches, and high-temperature sintered carbon such as petroleum or coal tar pitch derived cokes.

The binder attaches the active material to the conducting agent and fixes them on a current collector. The binder may include binders generally used in a lithium ion secondary battery. Examples of the binder may include polyvinylidene fluoride, polypropylene, carboxymethyl cellulose (CMC), polyvinylpyrrolidone, tetrafluoroethylene, polyethylene, polyvinyl alcohol, and styrene-butadiene rubber.

The conducting agent may be, for example, artificial graphite, natural graphite, acetylene black, ketjen black, channel black, lamp black, thermal black, conductive fiber such as carbon fiber or metallic fiber, conductive metal oxides such as titanium oxide, and metallic powder such as aluminum powder or nickel powder.

Examples of the separator may include a single olefin such as polyethylene (PE) and polypropylene (PP), or an olefin composite thereof, polyamide (PA), polyacrylonitrile (PAN), polyethylene oxide (PEO), polypropylene oxide (PPO), polyethylene glycoldiacrylate (PEGA), polytetrafluoroethylene (PTFE), polyvinylidene fluoride (PVdF), and polyvinylchloride (PVC).

The lithium secondary battery according to an embodiment may have any shape not limited to a specific shape. For example, the lithium secondary battery may be a cylindrical(can) type, a rectangular type, a pouch type, or a coin type.

One or more embodiments of the present disclosure will now be described in detail with reference to the following examples. However, these examples are only for illustrative purposes and are not intended to limit the scope of the one or more embodiments of the present disclosure.

<Preparation of Electrolyte Solution>

### Example 1

Ethylene carbonate, ethyl methyl carbonate, and dimethyl carbonate were mixed in a volume ratio of 2:4:4 to prepare an organic solvent. Next, LiPF₆ as a lithium salt was dissolved in the organic solvent to obtain a 1.15M LiPF₆ mixture solution. Next, 0.5 parts by weight of tetraethylammonium hexafluorophosphate as a solid salt, with respect to 100 parts by weight of the LiPF₆ mixture solution, was added, thereby preparing an electrolyte solution.

### Comparative Example 1

An electrolyte solution was prepared in the same manner as in Example 1, except that no solid salt was added.

<Manufacture of Battery>

LiNi_{0.5}Co_{0.2}Mn_{0.3}O₂ as a positive active material, polyvinylidene fluoride (PVdF) as a binder, and carbon black as a conducting agent were mixed in a weight ratio of 91.5:4.4:4.1, and then dispersed in N-methyl-2-pyrrolidone to prepare positive active material slurry. This slurry was coated on an aluminum current collector, dried, and roll-pressed to manufacture a positive electrode.

A graphite electrode was used as a negative electrode.

Next, the manufactured positive electrode, the negative electrode, and a porous polyethylene membrane (available from Tonen) as a separator were assembled into a coin cell, and a corresponding electrolyte solution prepared as above was injected thereinto.

<Evaluation Method>

1. Cell Formation

The coin cells prepared by using the electrolytes of Example 1 and Comparative Example 1 were left at a constant temperature of 25° C. for 12 hours, charged under conditions including a constant current of 0.1 C until a voltage was 4.3 V and a constant voltage having a terminating current of 0.05 C, and discharged under conditions including a constant current of 0.1 C until a voltage was 3.0 V by using a lithium secondary battery charger/discharger (TOSCAT-3600, available from Toyo-System Co., LTD), thereby completing a cell formation process.

2. Charge and discharge efficiency and high-temperature lifetime characteristics (%)

To evaluate lifetime characteristics, a charge and discharge capacity at 1^{st} cycle of each cell was measured by charging each cell after the cell formation with a constant current of 0.5 C until a voltage of 4.3V was reached, and then with a constant voltage with a cutoff voltage of 0.05 C, and discharging with a current of 0.5 C until a voltage of 3.0V was reached. This charge and discharge test at 45° C. was repeated 50 times under the same conditions. A charge and discharge efficiency and a capacity retention at a cycle were calculated using the following equations. The results are shown in Table 1.

Charge and discharge efficiency [%]=Discharge capacity/Charge capacity Capacity retention [%]=(Discharge capacity at 50^{th} cycle/Discharge capacity at 1^{st} cycle)×100

TABLE 1

| | 1^{st} cycle | | | 50^{th} cycle | | | |
|---|---|---|---|---|---|---|---|
| | Charge | Discharge | | Charge | Discharge | | Capacity |
| | capacity | capacity | Efficiency | capacity | capacity | Efficiency | retention |
| | (mAh/g) | (mAh/g) | (%) | (mAh/g) | (mAh/g) | (%) | (%) |
| Example 1 | 161.0 | 157.1 | 97.5 | 142.7 | 142.5 | 99.9 | 90.7 |
| Comparative | 161.5 | 158.2 | 97.9 | 140.6 | 140.0 | 99.6 | 88.5 |
| Example 1 | | | | | | | |

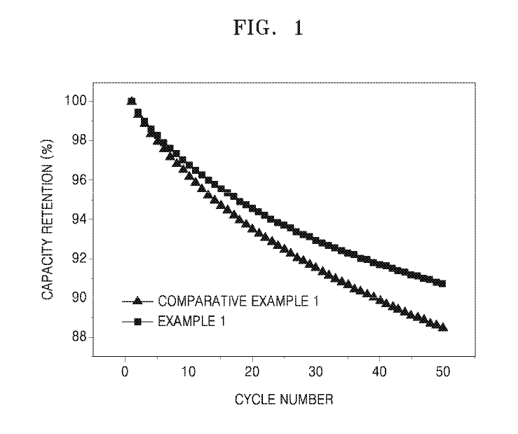

Referring to Table 1 and FIG. 1, the coin cell manufactured using the electrolyte solution of Example 1 was found to have improved charge and discharge capacities, improved charge and discharge efficiency, and improved lifetime characteristics after 50 times of charge and discharge cycles, compared to the coin cell manufactured using the electrolyte solution of Comparative Example 1.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope as defined by the following claims.

1. An electrolyte solution for a lithium secondary battery, the electrolyte solution comprising a lithium salt and an organic solvent,
wherein the electrolyte solution further comprises a solid salt represented by Formula 1: 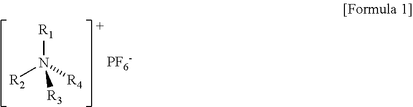
wherein, in Formula 1, R₁ to R₄ are each independently hydrogen, a halogen, or a C1 to C8 alkyl group.

2. The electrolyte solution of claim 1, wherein the solid salt represented by Formula 1 is at least one selected from the group consisting of ammonium hexafluorophosphate, tetramethylammonium hexafluorophosphate, tetraethylammonium hexafluorophosphate, tetrapropylammonium hexafluorophosphate, tetrabutylammonium hexafluorophosphate, tetrahexylammonium hexafluorophosphate, tetraheptylammonium hexafluorophosphate, ethyltrimethylammonium hexafluorophosphate, triethylmethylammonium hexafluorophosphate, butyltrimethylammonium hexafluorophosphate, diethyldimethylammonium hexafluorophosphate, and dibutyldimethylammonium hexafluorophosphate.

3. The electrolyte solution of claim 1, wherein an amount of the solid salt is in a range of 0.01 part to 5 parts by weight with respect to 100 parts by weight of a total weight of the lithium salt and the organic solvent.

4. The electrolyte solution of claim 1, wherein the lithium salt comprises at least one anion selected from the group consisting of F⁻, Cl⁻, Br⁻, I⁻, NO₃⁻, N(CN)₂⁻, BF₄⁻, ClO₄⁻, PF₆⁻, (CF₃)₂PF₄⁻, (CF₃)₃PF₃⁻, (CF₃)₄PF₂⁻, (CF₃)₅PF⁻, (CF₃)₆P⁻, CF₃SO₃⁻, CF₃CF₂SO₃⁻, (CF₃SO₂)₂N⁻, (FSO₂)₂N⁻, CF₃CF₂(CF₃)₂CO⁻, (CF₃SO₂)₂CH⁻, (SF₅)₃C⁻, (CF₃SO₂)₃C⁻, CF₃(CF₂)₇SO₃⁻, CF₃CO₂⁻, CH₃CO₂⁻, SCN⁻, and (CF₃CF₂SO₂)₂N⁻.

5. The electrolyte solution of claim 1, wherein the organic solvent is at least one selected from the group consisting of an ether, an ester, an amide, a linear carbonate, and a cyclic carbonate.

6. The electrolyte solution of claim 1, wherein the electrolyte solution comprises at least one selected from the group consisting of vinylene carbonate, vinyl ethylene carbonate, fluoroethylene carbonate, a cyclic sulfite, a saturated sultone, an unsaturated sultone, and a non-cyclic sulfone.

7. A lithium secondary battery comprising the electrolyte solution according to claim 1.
